(19) United States
(12) Patent Application Publication
(10) Pub. No.: US 2009/0170900 A1
(43) Pub. Date: Jul. 2, 2009

(54) DOSING REGIMEN FOR WEIGHT LOSS

(75) Inventors: Henry Sjoerd Koopmans, Calgary (CA); Keith Alexander Sharkey, Calgary (CA); Adam Paul Chambers, Calgary (CA)

Correspondence Address: BENNETT JONES LLP;C/O MS ROSEANN CALDWELL, 4500 BANKERS HALL EAST, 855 - 2ND STREET, SW, CALGARY, AB, T2P 4K7, CA

(73) Assignee: UTI LIMITED PARTNERSHIP, Calgary, AB (CA)

(21) Appl. No.: 12/063,024

(22) PCT Filed: Aug. 4, 2006
(86) PCT No.: PCT/CA06/01284

§ 371 Date: Jul. 22, 2008

(30) Foreign Application Priority Data
Sep. 5, 2005 (CA) 2518579

(51) Int. Cl. A61K 31/454; A61P 3/04

(52) U.S. Cl. 514/326

(57) ABSTRACT

Disclosed are dosing regimens and methods for the treatment of obesity, overweight and/or overeating in mammals comprising administering to the mammal a pharmaceutically effective amount of a CB1 receptor antagonist as a unit dosage according to a continuous schedule having a dosing interval selected from the group consisting of once-weekly dosing, twice-weekly dosing, thrice-weekly dosing, biweekly and bimonthly.

This application claims the benefit of U.S. Provisional Patent No. 60/595,791, filed Aug. 5, 2005 and Canadian Patent Application No. 2,518,579, filed Sep. 8, 2005.

### FIELD OF THE INVENTION

The present invention relates to dosing regimens and methods for the treatment of obesity, overweight and/or overeating in mammals comprising administering to the mammal a pharmaceutically effective amount of a CB₁ receptor antagonist as a unit dosage according to a continuous schedule having a dosing interval selected from the group consisting of once-weekly dosing, twice-weekly dosing, thrice-weekly dosing, biweekly dosing and bimonthly dosing.

More particularly, the present invention relates to a method for reducing food intake and/or body weight in a mammal comprising administering to said mammal a pharmaceutically effective amount of a CB₁ receptor antagonist once every 2 to 14 day or, in the alternative, on two or three consecutive days every 7 to 14 days, preferably prior to or during a period of increased caloric intake.

### BACKGROUND OF THE INVENTION

Obesity is widely recognized as a serious health problem that is increasing in prevalence across the United States and the world. According to the 1998 National Institute of Health (NIH) Clinical Guidelines on the Identification, Evaluation and Treatment of Overweight and Obesity in Adults, an estimated 97 million people in the US are classified as either overweight or obese. The medical and other costs related to obesity have risen considerably in the last two decades (K. M. Flegal et al. 2005, “Excess deaths associated with underweight, overweight and obesity.” JAMA 293: 1861-7). In addition, many pets or companion animals, such as dogs or cats, have become obese and their owners may seek veterinary treatment to cure their obesity and associated medical problems.

Agents that have been or are currently being used for the treatment of obesity include phenylpropanolamine, dexfenfluramine, phentermine/tenfluramine, sibutramine and orlistat (L. M. Kaplan, 2005. “Pharmacological Therapies for Obesity” Gastro Clin N Am 34: 91-104.). Unfortunately, all of these drugs have serious adverse effects and dexfenfluramine and fenfluramine have been withdrawn because of toxicity associated with valvular heart disease in a small subset of patients (H. M. Connolly et al., 1997. “Valvular Heart Disease associated with Fenfluramine-phentermine.” N. Engl J Med 337: 581-8; F. F. Seghatol and V. H. Rigolin, 2002. “Appetite Suppressants and Valvular Heart Disease.” Curr Opin Cardiol 17: 486-93). Thus, there is a therapeutic need for safer and more effective compounds to treat obesity.

Much attention has been focused in the last ten years on the endocannabinoid system for its potential for pharmacologic manipulation to treat obesity. The endocannabinoid system comprises endogenous ligands commonly referred to as cannabinoids (anandamide, 2-arachidonoyl glycerol, 2-arachidonyl glyceryl ether (noladin ether), virodhamine), and two cannabinoid receptor subtypes (CB₁ and CB₂). Marijuana and the major plant cannabinoid, delta(9)tetrahydrocannabinoid, have been implicated in the changes of feeding behavior in both man and animals. Recently, researchers have shown that in partially satiated animals, the administration of the naturally occurring cannabinoids anandamide and 2-arachidonyl glycerol increases food intake. It is believed that these endocannabinoids stimulate the CB₁ and CB₂ receptors, which alter glucose and lipid metabolism in both liver and adipose tissue and, most notably, help to regulate energy balance and body weight.

The cannabinoid CB₁ receptor has received the greatest attention with respect to appetite and body weight regulation, leading to the development of a new class of appetite suppressants and/or weight regulating drugs that appear to work by selectively blocking the CB₁ receptors. The discovery of the first selective CB₁ receptor antagonist was reported several years ago. See M. Rinaldi-Carmona et al., 1994, “SR141716A, a Potent and Selective Antagonist of the Brain Cannabinoid Receptor”, FEBS Letters 350: 240-244. This antagonist compound, N-(piperidin-1-yl)-5-(4-chlorophenyl)-1-(2,4-dichlorophenyl)-4-methyl-1H-pyrazole-3-carboxamide (SR141716A or rimonabant), has been shown to have anorectic efficacy and produce a sustained reduction in body weight. SR141716A is the hydrochloride of SR 141716. See G. Columbo et al., 1998, “Appetite Suppression and Weight Loss After Cannabinoid Antagonist SR 141716”, Life Sci 63:PL 113-7 and U.S. Pat. No. 6,344,474, incorporated herein by reference.

It is theorized that SR141716A binds to CB₁ receptors and competitively antagonizes many of the CB₁ receptor-mediated effects of cannabinoids. Thus, synthesis of an antagonist such a SR141716A that selectively binds to CB₁ receptors without producing cannabimimetic activity in vivo suggests that recognition and activation of cannabinoid receptors are separable events. See D. R. Compton et al., 1996, “In Vivo Characterization of a Specific Cannabinoid Receptor Antagonist (SR141716A): Inhibition of delta 9-tetrahydrocannabinol-induced Responses and Apparent Agonist Activity”, J Pharmacol Exp Ther. 277: 586-594.

It was further discovered that administration of the compound N-(piperidin-1-yl)-5-(4-iodophenyl)-1-(2,4-dichlorophenyl)-4-methyl-1H-pyrazole-3-carboxamide (AM-251) also reduced food intake. See R. Z. Chen et al., 2004, “Synergistic Effects of Cannabinoid Inverse Agonist AM-251 and Opioid Antagonist Nalmefene on Food Intake and Body Weight Gain of Obese (fa/fa) Compared to Lean Zucker Rats”, Psychopharmacology (Berl) 167: 103-11 and A. L. Hildebrandt et al., 2003, “Antiobesity Effects of Chronic Cannabinoid CB1 Receptor Antagonist Treatment in Diet-induced Obese Mice”, Eur J Pharmacol 462:125-32. Other compounds studied to date include N-(Morpholin-1-yl)-1-(2,4-dichlorophenyl)-5-(4-iodophenyl)-4-methyl-1H-pyrazole-3-carboxamide (AM 281) (see R. Lan et al., 1999, “Design and Synthesis of the CB1 Selective Cannabinoid Antagonist AM 281: A Potential Human SPECT Ligand”, AAPS Pharmasci 1(3) article 4) and 3-(4-chlorophenyl-N′-(4-chlorophenyl)sulfonyl-N-methyl-4-phenyl-4,5-dihydro-1H-pyrazole-1-carboxamide (SLV-319) (see J. H. Lange et al., 2004, “Synthesis, Biological Properties, and Molecular Modeling Investigations of Novel 3,4-diarylpyrazolines as Potent and Selective CB(1) Cannabinoid Receptor Antagonists”, J Med Chem. 47(3): 627-43.), both of which were found to be potent, CB₁ selective antagonists. For a review of CB₁ cannabinoid receptor antagonists, see J. H. M. Lange and C. G. Kruse, 2005, “Medicinal Chemistry Strategies to CB₁ Cannabinoid Receptor Antagonists”, DDT 10(10): 693-702.

The Columbo et al. study showed that food intake was quickly decreased with rimonabant-treated rats when compared with placebo-treated rats. However, as treatment continued, tolerance or desensitization to the suppressant effect of rimonabant developed. The waning of the anorectic effect was shown to occur after only 5 days of treatment. Similarly, in the Hildebrandt et al. study, the anorectic efficacy of AM-251 also waned over time during treatment. Repeated daily administrations of AM-251 (3 mg/kg) were shown to lead to non-significant changes in food intake after only 8 days in mouse. Further, both the Columbo et al. and the Hildebrandt et al. studies suggest that when daily doses of SR141716A and AM-251 (approximately 10 mg/kg) are stopped, a significant rebound hyperphagia and a rapid return in body weight occurs.

Finally, there are potential side effects associated with treatment with CB₁ antagonists. In human trials, the most common side effects of SR141716A (5 and 20 mg doses) were nausea, dizziness, arthralgia and diarrhea (L. F. van Gaal et al., 2005. “Effects of the CB1 receptor Blocker Rimonabant on weight Reduction and cardiovascular risk factors in overweight patients: 1-year experience from the RIO-Europe Study.” Lancet 365: 1389-97). These events were considered to be mild or moderate in intensity and transient based on occurrence early in the study. Further, mice treated with SR141716A showed significantly more damage in response to an inflammatory stimulus compared with vehicle-treated controls. See F. Massa et al., 2004, “The Endogenous Cannabinoid System Protects Against Colonic Inflammation”, J Clin Invest 1202-9; G Kunos and P Pacher, 2004. “Cannabinoids Cool the Intestine.” Nature Medicine 10: 678-9. In addition, in an animal model of temporal lobe epilepsy, treatment with SR141716A led to a significantly higher frequency of protracted seizures. See M. J. Wallace et al., 2003, “The Endogenous Cannabinoid System Regulates Seizure Frequency and Duration in a Model of Temporal Lobe Epilepsy”, J Pharmacol Exp Ther 307: 129-37.

It is clear from the current literature that continuous daily treatment of obesity by daily administration of CB₁ receptor antagonists such as rimonabant and AM-251 has shortcomings, and that there is a need for development of a dosing regimen to overcome these shortcomings.

In the present invention, it is surprisingly found that a single, relatively low dose of CB₁ receptor antagonist can result in long-term effects on food intake. Food intake is significantly reduced for six days or more following a single administration. Thus, it is found that by administering a CB₁ receptor antagonist according to a continuous schedule having a dosing interval of once-weekly dosing, twice-weekly dosing, or thrice-weekly dosing, many of the adverse effects observed with daily doses of CB₁ receptor inhibitors can be minimized. In particular, such a dosing regimen reduces the development of tolerance or desensitization to the suppressant effect of the CB₁ receptor antagonist. Further, administration of the antagonist at a relatively low dosage at a relatively low dosing frequency is less likely to cause significant rebound-hyperphagia. Finally, weekly or twice- or thrice-weekly dosing or biweekly dosing or bimonthly dosing could minimize other unwanted side effects observed with CB₁ receptor antagonists.

From a patient lifestyle standpoint, the methods of the present invention would also be more convenient than daily regimens and would result in greater patient compliance. The patient could focus on a particular time of the week when the medicine would be taken most effectively. Further, in view of the observation that CB₁ receptor antagonists are most active in the first three days, dosing regimens could be commenced on the day before periods of rest and socialization (such as week-ends and the like), which have been shown to be a period of time when people overeat by about 8-13%. See S. T. St. Jeor et al. 1983 “Variability in Nutrient Intake in a 28-day Period.” J. Amer Diet Assoc 83:155-62; J. M. De Castro, 1991, “Weekly Rhythms of Spontaneous Nutrient Intake and Meal Pattern of Humans”, Physiology & Behavior 50: 729-738; J. M. de Castro, 2000. “Eating Behavior: Lessons from the Real World of Humans” Nutrition 16: 800-13.

Thus, by way of example, a patient can commence the weekly regimen on a Friday, before the Saturday/Sunday weekend that is enjoyed as a period of rest and socialization by much of the Western world.

### SUMMARY OF THE INVENTION

In one aspect, the invention herein relates to the surprising finding that the weight loss effect of CB₁ receptor antagonists continues far longer than 1-2 days after administration. A reasonable concentration of the drug might be expected to remain within the brain for 1-2 days. In the present invention, it is found that reductions in body weight and/or food intake can occur for a period of at least 3-6 days after administration of the CB₁ receptor antagonist and may continue even longer. This surprising result would not have been predicted based on the known pharmacokinetics of CB₁ antagonists. For example, the duration of the weight loss and/or anorectic effect of rimonabant or AM-251 far exceeded the pharmacological profile described in mouse brain, wherein intravenously injected ¹²³I-labeled AM-251 radioactivity declined to about half its peak level after only 8 hours in mouse brain. See S. J. Gatley et al., 1996, “¹²³I-labled AM251: a Radioiodinated Ligand Which Binds in vivo to Mouse Cannabinoid CB₁ Receptors”, Eur J Pharmacol 307: 331-8, incorporated herein by reference.

The anorectic and/or weight loss effect of the present invention also exceeded the predicted half-life for rimonabant or AM-251 of 22 hours, as determined from behavioral tests. See P. J. McLaughlin, et al., 2003, “The cannabinoid CB₁ antagonists SR 171416A and AM-251 suppress food intake and food reinforced behavior in a variety of tasks in rats.” Behav Pharmacol 14: 583-8. In addition, in the present invention it was found that daily food intake was significantly reduced for several days, even though a 48-hr test for the continued presence of the CB₁ antagonist, AM-251, in the brain showed that AM-251 was no longer present in the brain in sufficient concentrations to eliminate the well-known hypothalamic suppression of body temperature by delta-THC, a cannabinoid agonist.

In the present invention, the reduction in weight loss may be accompanied by a reduction in food intake in a dose dependent manner. Surprisingly, in the present invention, the duration of weight loss, the most important measure, exceeded the duration of reduced food intake. In particular, when animals received the lowest dose of the drug, the termination of the delivery of CB₁ receptor antagonists has been shown to lead to significant hyperphagia and a rapid return in body weight when treatment is stopped. Therefore, continued infrequent use of the drug is an effective way to prevent the regain of lost body weight.

Thus, in one aspect, the invention herein relates to methods for the treatment of obesity, overweight and/or overeating in a mammal in need thereof, comprising administering to said mammal a pharmaceutically effective amount of a CB₁ receptor antagonist as a unit dosage according to a continuous schedule having a dosing interval selected from the group consisting of once-weekly dosing, twice-weekly dosing, thrice-weekly dosing, biweekly dosing and bimonthly dosing, wherein said continuous schedule is maintained until the desired therapeutic effect is achieved for said mammal.

In other embodiments, the invention relates to methods comprising a continuous dosing periodicity ranging from about once every 2 days to about once every 14 days.

In another aspect, the invention relates to methods for reducing food intake and/or body weight in a mammal in need of such treatment. More particularly, the invention relates to methods for reducing food intake and/or body weight in a mammal in need of such treatment comprising administering a single dose of a pharmaceutically effective amount of a CB₁ receptor antagonist prior to an anticipated period of higher food intake (e.g., higher caloric intake). In one embodiment, the invention relates to methods for reducing food intake and/or body weight in a mammal in need of such treatment comprising administering a pharmaceutically effective amount of a CB₁ receptor antagonist for two or three consecutive days every 7 to 14 days, preferably prior to or during a period of increased caloric intake.

In other embodiments, the invention herein relates to such methods useful in humans.

In other embodiments, the invention herein relates to such methods useful in companion animals, such as dogs or cats.

The present invention may result in one or more of the following effects:
(1) a lowering of the overall dosage of the CB₁ receptor antagonist drug needed to cause a fixed amount of weight loss;
(2) a dosing regimen of once-weekly dosing, twice-weekly dosing and thrice-weekly dosing which leads to an equal to or greater loss of weight;
(3) a more sustained weight loss and longer prevention of weight regain;
(4) a reduction in weekly food intake;
(5) an increase in weekly energy expenditure (EE);
(6) a reduction in the amount of tolerance that develops for the antagonist drug, as measured by the combination of reduced daily food intake, increased EE and sustained body weight loss;
(7) a reduction in the adverse side effects of the antagonist drug, including possible nausea, dizziness, arthralgia and diarrhea, seen in a small percentage of the patients;
(8) an improvement in the characteristics of the metabolic syndrome commonly found in obese individuals, including improved glucose tolerance, blood lipid levels and blood pressure levels; and
(9) a reduction in the amount of inflammation or other physiological or psychological damage (if such damage occurs in some patients) resulting from continuous use of the antagonist drug.

### BRIEF DESCRIPTION OF THE DRAWINGS

FIG. 1 is a bar graph showing cumulative food intake over 3 h after injection of either vehicle or CB₁ receptor antagonist AM-251.

FIG. 2 shows the changes in daily food intake starting 2 days before and finishing 7 days after administration of a single dose of either vehicle or 5.0 mg/kg CB₁ receptor antagonist AM-251.

FIG. 3 shows the dose-dependent changes in daily food intake (g) starting 2 days before and finishing 7 days after administration of a single dose of either vehicle or CB₁ receptor antagonist AM-251 (1.25 mg/kg or 2.5 mg/kg).

FIG. 4 shows the changes in weight gain or loss (g) starting 2 days before and finishing 8 days after administration of a single dose of either vehicle or CB₁ receptor antagonist AM-251 (1.25 mg/kg or 2.5 mg/kg).

FIG. 5 shows the daily food intake (mean±SEM; g) expressed in g whereby one gram equals 1.41 kcal for vehicle treated rats (filled triangles) and rats treated every 5 days with AM-251 (5 mg/kg) (hollow circles). Hash marks on the x-axis indicate where 5 mg/kg treatments were given. Significant differences in food intake between vehicle- and AM-251-treated rats are shown by the bars, p<0.05, unpaired t-test.

FIG. 6 shows the changes in body weight (mean±SEM; g) starting the day before treatment with either vehicle (filled triangles), or AM-251 (5 mg/kg every 5 days, hollow circles). Hash marks on the x-axis indicate where 5 mg/kg treatments were given.

FIG. 7 shows the average daily body weight of male rats given daily or twice-weekly injections of the cannabinoid antagonist, AM-251.

FIG. 8 shows the average daily body weight of male rats given daily or twice-weekly injections of the cannabinoid antagonist, rimonabant.

### DETAILED DESCRIPTION OF THE INVENTION

The present invention relates to a method, preferably an oral method, for treating disorders of feeding behavior or energy balance, such as obesity, overweight and/or overeating, comprising administering to a subject in need thereof a therapeutically effective amount of a CB₁ receptor antagonist.

CB₁ receptor antagonists of the present invention are broadly defined as those compounds that selectively bind to CB₁ receptors without producing cannabimimetic activity in vivo.

One genus of compounds useful in the present invention correspond to the chemical formula:

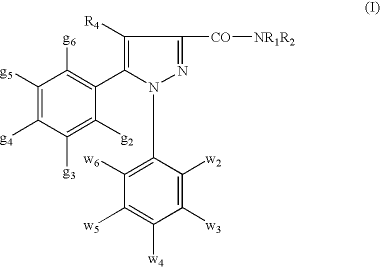

in which:
g₂, g₃, g₄, g₅ and g₆, and w₂, w₃, w₄, w₅ and w₆, are identical or different and are independently hydrogen, a chlorine, bromine or iodine atom, a (C₁-C₃)alkyl, a (C₁-C₃)alkoxy, a trifluoromethyl or a nitro group and g₄ can also be a phenyl group;
R₁ is a (C₁-C₆)alkyl or a hydrogen;

### R₂ is —⁺NR₃R₅R₆ or —NR₅R₆;

R₃ is a (C₁-C₆)alkyl or R₃ forms a bridge with one of the atoms of the heterocyclic radical formed by NR₅R₆;
R₄ is hydrogen or a (C₁-C₅)alkyl; and
R₅ is hydrogen or a (C₁-C₆)alkyl and R₆ is hydrogen, a (C₁-C₆)alkyl, a phenyl or a (C₃-C₈)cycloalkyl, or R₅ and R₆, together with the nitrogen atom to which they are bonded, form a 5- to 10-membered saturated or unsaturated heterocyclic radical which is unsubstituted or monosubstituted or polysubstituted by a (C₁-C₆)alkyl, a benzyl, a phenyl, a hydroxyl, a (C₁-C₆)alkoxy or a halogen, with the proviso that if R₂ is NR₅R₆, R₅ and R₆, together with the nitrogen atom to which they are bonded, form a heterocyclic radical other than a 5- to 8-membered saturated radical which is unsubstituted or substituted by a (C₁-C₃)alkyl, a hydroxyl or a benzyl, their salts or their solvates.

(C₁-C₃)alkyl, (C₁-C₅)alkyl and (C₁-C₆)alkyl are understood as meaning C₁-C₃, C₁-C₅ and C₁-C₆ linear or branched alkyls. The preferred alkyl groups are methyl, ethyl, propyl and isopropyl groups.

5- or 10-membered saturated or unsaturated heterocyclic radical is understood as meaning a fused or bridged, mono-, di- or tri-cyclic, non-aromatic heterocyclic radical which can contain a second heteroatom such as nitrogen, oxygen or sulfur. These radicals include the following radicals in particular: pyrrolidin-1-yl, piperidin-1-yl, hexahydroazepin-1-yl, morpholin-4-yl, thiomorpholin-4-yl, 2-azabicyclo[2.2.2]oct-5-en-2-yl, 2-methyl-2-azoniabicyclo[2.2.2]oct-5-2-yl, 2-azaadamant-2-yl, 1,2,3,6-tetrahydropyridin-1-yl, 2-azabicyclo[2.2.1]heptan-2-yl, 2-azabicyclo[2.2.2]octan-2-yl and 1-azoniabicyclo[2.2.2]octan-1-yl.

The salts of the compound of formula (I) include the pharmaceutically acceptable acid addition salts such as the hydrochloride, hydrobromide, sulfate, hydrogen sulfate, dihydrogen phosphate, methanesulfonate, methylsulfate, maleate, oxalate, fumarate, naphthalene-2-sulfonate, glyconate, gluconate, citrate, isethionate, paratoluenesulfonate and mesitylenesulfonate salt.

Among the compounds of formula (1), N-(piperidin-1-yl)-5-(4-chlorophenyl)-1-(2,4-dichlorophenyl)-4-methyl-1H-pyrazole-3-carboxamide (also known as rimonabant) and N-(piperidin-1-yl)-5-(4-iodophenyl)-1-(2,4-dichlorophenyl)-4-methyl-1H-pyrazole-3-carboxamide (also known as AM-251), and their pharmaceutically acceptable salts are particularly advantageous.

Other non-limiting examples of CB₁ receptor antagonists according to the present invention are 5-(4-bromophenyl)-1-(2,4-dichlorophenyl)-4-ethyl-N-(1-piperidinyl)-1H-pyrazole-3-carboxamide; 3-(4-chlorophenyl-N′-(4-chlorophenyl)sulfonyl-N-methyl-4-phenyl-4,5-dihydro-1H-pyrazole-1-carboxamide; the compounds described in U.S. Pat. No. 5,462,960, to Barth et al., issued Oct. 31, 1995; the compounds described in U.S. Pat. No. 5,624,941, to Barth et al., issued Apr. 29, 1997; the compounds described in U.S. Pat. No. 6,028,084, to Barth et al., issued Feb. 22, 2000; the compounds described in U.S. Pat. No. 6,432,984, to Barth et al., issued Aug. 13, 2002; the compounds described in U.S. Pat. No. 6,509,367, to Martin et al., issued Jan. 21, 2003; the alkyl amide compounds disclosed in U.S. Pat. No. 6,825,209, to Thomas et al., issued Nov. 30, 2004; the compounds disclosed in U.S. Pat. No. 5,747,524, to Cullinan et al., issued May 5, 1998; the compounds disclosed in U.S. Pat. No. 5,596,106, to Cullinan et al., issued Jan. 21, 1997; the substituted pyrazole compounds described in International Application No. PCT/IB2004/001484, published Nov. 18, 2004 as WO 2004/099157; the bicyclic pyrazolyl and imidazolyl compounds described in International Application No. PCT/IB2004/001262, published Nov. 4, 2004 as WO 2004/094417; the compounds described in International Application No. PCT/IB2004/001357, published Nov. 4, 2004 as WO 2004/094421; the compounds described in International Application No. PCT/IB2004/001482, published Nov. 4, 2004 as WO 2004/094429; the imidazole compounds described in International Application No. PCT/IB2004/002442, published Feb. 3, 2005 as WO 2005/009974; the azetidine derivatives as cannabinoid antagonists disclosed in U.S. Pat. No. 6,355,631, U.S. Pat. No. 6,479,479 and PCT publications WO 01/64632, 01/64633, and 01/64634; the substituted 3-alkyl and 3-alkenyl azetidine derivatives described in International Application No. PCT/US2004/018348, published Jan. 6, 2005 as WO 2005/000809; the azetidine-1-carboxamides described in International Application No. PCT/GB2004/001831, published Nov. 11, 2004 as WO 2004/096763; the azetidine-1-carboxamides described in International Application No. PCT/GB2004/001812, published Nov. 11, 2004 as WO 2004/096209; the bi-heteroaryl compounds described in International Application No. PCT/IB2003/004411, published Apr. 29, 2004 as WO 2004/035566; tetrahydroquinoline containing compounds described in International Application No. PCT/US2004/022408, published Jan. 27, 2005 as WO 2005/007628; and substituted pyrazolopyridazine and imidazopyridazine compounds described in U.S. patent application Ser. No. 10/853,993, published Dec. 9, 2004 as US 2004/0248881, all of which are incorporated herein by reference.

Compounds of the present invention include pharmaceutically acceptable salts of the compounds and hydrates or solvates of the compounds.

The term “pharmaceutically effective amount”, as used herein, means that amount of the CB₁ receptor antagonist that will elicit the desired therapeutic effect or response when administered in accordance with the desired treatment regimen. A preferred pharmaceutically effective amount of the CB₁ receptor antagonist is an appetite-suppressing amount and/or an amount that results in weight loss.

By “once-weekly dosing” is meant that a unit dosage of the CB₁ receptor antagonist is administered once a week, i.e., one time during a seven-day period, preferably on the same day of each week. In the once-weekly dosing regimen, the unit dosage is generally administered about every seven days. A non-limiting example of a once-weekly dosing regimen would entail the administration of a unit dosage of the CB₁ receptor antagonist every Friday.

By “twice-weekly dosing” is meant that a unit dosage of the CB₁ receptor antagonist is administered twice a week, i.e., two times during a seven-day period, preferably on the same two days of each weekly period. In the twice-weekly dosing regimen, each unit dosage may be either administered every three to four days or delivered on two consecutive days followed by a rest period of four to five days. A non-limiting example of a twice-weekly dosing regimen would entail the administration of a unit dosage of the CB₁ receptor antagonist every Friday and Tuesday. Another non-limiting example would entail the administration of the CB₁ receptor antagonist on two consecutive days, preferably either prior to or during a period of increased caloric intake, for example, prior to a “weekend”.

By “biweekly dosing” is meant that a unit dosage of the CB₁ receptor antagonist is administered once during a two-week period, i.e., one time during a fourteen-day period, preferably on the same day during each two-week period. In the twice-weekly dosing regimen, each unit dosage is generally administered about every fourteen days. A non-limiting example of a biweekly dosing regimen would entail the administration of a unit dosage of the CB₁ receptor antagonist every other Friday.

By “twice-monthly dosing” is meant that a unit dosage of the CB₁ receptor antagonist is administered twice, i.e., two times, during a monthly calendar period. With the twice-monthly regimen, the doses are preferably given on the same two dates of each month. In the twice-monthly dosing regimen, each unit dosage is generally administered about every fourteen to sixteen days. A non-limiting example of a biweekly dosing regimen would entail dosing on or about the first of the month and on or about the fifteenth, i.e., the midway point, of the month.

Compositions of the present invention comprise a pharmaceutically effective amount of a CB₁ receptor antagonist compound. The CB₁ receptor antagonist compounds of the present invention can be administered in the form of any pharmaceutical formulation, the nature of which will depend upon the route of administration. These pharmaceutical compositions can be prepared by conventional methods, using compatible, pharmaceutically acceptable excipients or vehicles. Examples of such compositions include capsules, tablets, transdermal patches, lozenges, troches, sprays, syrups, powders, granulates, gels, elixirs, suppositories, and the like, for the preparation of extemporaneous solutions, injectable preparations, rectal, nasal, ocular, vaginal etc. A preferred route of administration is the oral route.

For oral administration, tablets containing various excipients such as microcrystalline cellulose, sodium citrate, calcium carbonate, dicalcium phosphate and glycine may be employed along with various disintegrants such as starch (preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc can be used for tabletting purposes. Solid compositions of similar type may also be employed as fillers in gelatin capsules; preferred materials in this connection also include lactose or milk sugar, as well as high molecular weight polyethylene glycols. When aqueous suspensions and/or elixirs are desired for oral administration the active ingredient may be combined with sweetening or flavoring agents, coloring matter and, if so desired, emulsifying and/or suspending agents, together with such diluents as water, ethanol, propylene glycol, glycerin and various combinations thereof.

The dosage form can be designed for immediate release, controlled release, extended release, delayed release or targeted delayed release. The definitions of these terms are known to those skilled in the art. Furthermore, the dosage form release profile can be effected by a polymeric mixture composition, a coated matrix composition, a multiparticulate composition, a coated multiparticulate composition, an ion-exchange resin-based composition, an osmosis-based composition, or a biodegradable polymeric composition. Without wishing to be bound by theory, it is believed that the release may be effected through favorable diffusion, dissolution, erosion, ion-exchange, osmosis or combinations thereof.

For parenteral administration, a solution of a CB₁ receptor antagonist compound in either sesame or peanut oil or in aqueous propylene glycol can be employed. The aqueous solutions should be suitably buffered (preferably pH greater than 8), if necessary, and the liquid diluent first rendered isotonic. The aqueous solutions are suitable for intravenous injection purposes. The preparation of all these solutions under sterile conditions is readily accomplished by standard pharmaceutical techniques well known to those skilled in the art.

The precise dosage of the CB₁ receptor antagonist may vary with the dosing schedule, the oral potency of the particular CB₁ receptor antagonist chosen, the age, size, sex and condition of the mammal or human, and other relevant medical and physical factors. Generally, however, for humans, the CB₁ receptor antagonists is administered in dosages ranging from about 0.5 to about 100 mg, advantageously from 1 to 40 mg and preferably from 2 to 35 mg per dosage unit, for weekly or twice- or thrice-weekly administration. For biweekly or twice-monthly dosing, the dosages may be moderately higher and preferably 5 to 50 mg per dosage unit.

The following non-limitative examples further describe and enable a person ordinarily skilled in the art to make and use the invention.

### EXAMPLE 1

### Early Effects on Food Intake of N-(Piperidin-1-yl)-5-(4-iodophenyl)-1-(2,4-dichlorophenyl)-4-methyl-1H-pyrazole-3-carboxamide (AM-251) in Rats

The experiment is performed according to A. P. Chambers et al., 2004, “Cannabinoid (CB)1 Receptor Antagonist, AM-251, Causes a Sustained Reduction of Daily Food Intake in the Rat”, Physiology & Behavior 82: 863-869, incorporated herein by reference.

Briefly, moderately obese male Lewis rats (n=8), weighing between 440 and 500 g at the beginning of the study, were individually housed in opaque plastic cylinder cages in a temperature-controlled environment between 20 and 22° C., under a 12:12 h light-dark cycle (lights off at 1800 h). Vanilla-flavoured Ensure Plus (Ross Laboratories, Saint-Laurent, Quebec, Canada) was used as food on account of its palatability, and to reduce the risk of spillage. A milky-sweet liquid, Ensure Plus is composed of 53.3% carbohydrate, 29% fat, 16.7% protein (1.41 kcal/g), which includes daily minerals and vitamins typical of a Western diet. Food was available from 1600 to 1700 (prefeed) and 1800-0900 h daily; water was freely available at all times. Food and water were presented in inverted glass bottles that attached to the outside of the cage. Animals were habituated to handling and testing procedures 3 weeks prior to testing on liquid diet.

The initial food presented between 1600 and 1700 h (prefeed) was designed to produce partial satiety as previously described in C. M. Williams and T. C Kirkham, 1999, “Anandamide Induces Overeating: Mediation by Central Cannabinoid (CB1) Receptors”, Psychopharmacology (Berl) 143: 315-7 and R. Gomez, 2002, “A Peripheral Mechanism for CB1 Cannabinoid Receptor-Dependent Modulation of Feeding”, J Neurosci 22: 9612-7, both papers incorporated herein by reference.

AM-251 (N-(piperidin-1-yl)-5-(4-iodophenyl)-1-(2,4-dichlorophenyl)-4-methyl-1H-pyrazole-3-carboxamide) was purchased from Tocris Cookson and resuspended in a vehicle (2% dimethyl sulfoxide, 1% Tween 80 and 97% physiological saline). Drugs were aliquoted and placed in a freezer at −70° C. until use.

At approximately 17:45 h, rats were given an intraperitoneal injection of either AM-251 (n=7) at 5 mg/kg or vehicle (n=8). Food bottles were put back on at 1800 h. Food intake measurements were taken daily as well as at 1900, 2000, and 2100 h.

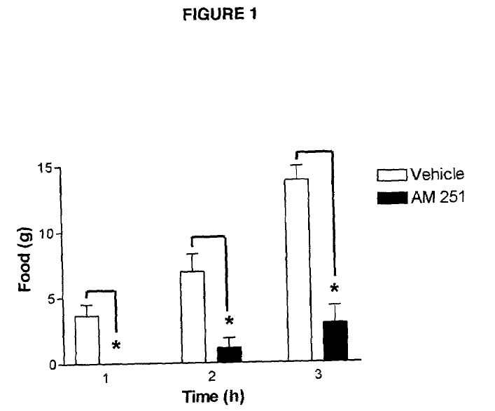

FIG. 1 shows the cumulative food intake for AM-251 and vehicle-control conditions after 1 hour, 2 h and 3 h of testing. Rats that were given AM-251 ate significantly less than the vehicle control rats at all three time points [1 h: AM-251 no intake vs. control 3.6±0.8 g; q(6)=4.01; P<0.01; 2 h: 1.3±0.8 g vs. 6.9±0.8; q(6)=2.86; P<0.05; 3 h: 3.3±1.4 g vs. 13.8±3.3; q(6)=19.75; P<0.01].

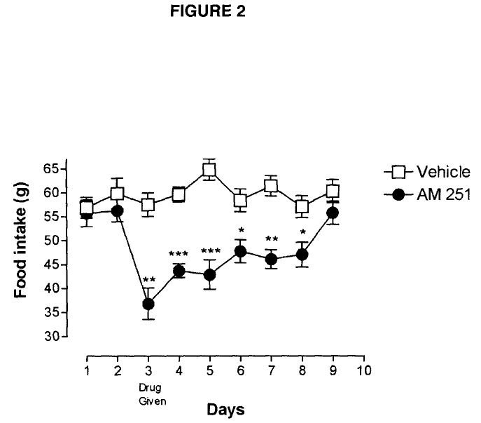

FIG. 2 is a plot of the changes in daily food intake (g), measured starting 2 days prior to administration and finishing 7 days after administration, of either vehicle or AM-251 (5 mg/kg). It can be seen from FIG. 2 that there was a prolonged reduction in food intake after a single administration of AM-251. The reduction in daily food intake brought about by AM-251 was significant for a total of 6 days, starting on the day of injection [AM-251: 36.7±3.3 g vs. control: 57.4±2.4; t(6)=4.13; P=0>003], and finishing the day before the next drugs were given [AM-251: 47.0±2.5 g vs. control: 57.0±2.2 g; t(6)=2.53; P=0.022].

### EXAMPLE 2

### Long Term Effect of N-(Piperidin-1-yl)-5-(4-iodophenyl)-1-(2,4-dichlorophenyl)-4-methyl-1H-pyrazole-3-carboxamide (AM-251)

The original protocol described in Example 1 was repeated using two additional doses (1.25 mg/kg or 2.5 mg/kg) of the CB₁ receptor antagonist AM-251 or vehicle. Food intake and weight measurements were taken daily. Naïve male Lewis rats (n=8), weighing between 469 and 520 g at the beginning of the study, were individually housed in plastic cages, in a temperature-controlled environment between 20 and 22° C., under a 12:12 h light-dark cycle (lights off 1900 h). Food was available from 1600 to 1700 (prefeed) and 1800-900 h daily; water was freely available at all times. Animals were assigned into vehicle, 1.25 or 2.5 mg/kg conditions via a counterbalance design. AM-251 was supplied and administered as previously described at 1745 h.

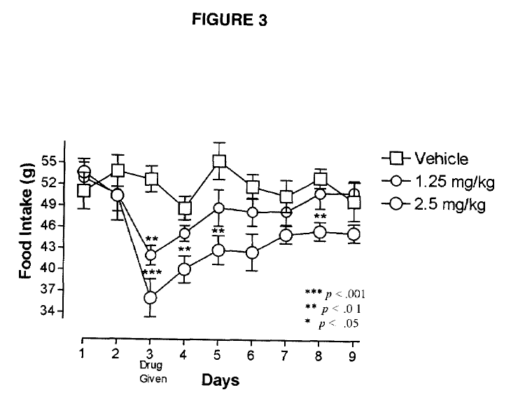

FIG. 3 shows the dose-dependent changes in daily food intake (g) starting 2 days prior to administration of the drug or vehicle and finishing 7 days after administration of either vehicle or AM-251 (1.25 mg/kg or 2.5 mg/kg). As can be seen from the graph, reductions in food intake brought about by AM-251 were dose dependent. Reductions in food intake using 2.5 mg/kg of AM-251 were significant starting the day of administration and for the next 4 days p<0.001.

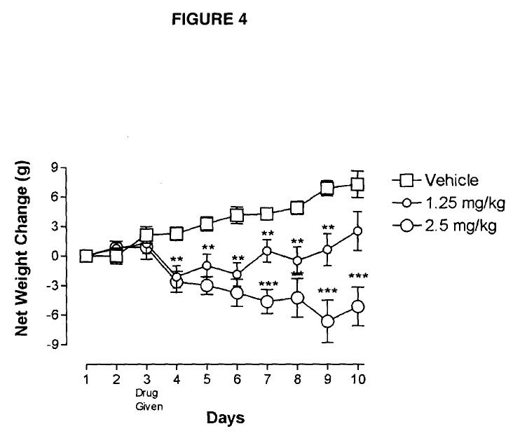

FIG. 4 shows the changes in weight gain or loss (g) starting 2 days before and finishing 8 days after the administration of either vehicle or AM-251 (1.25 mg/kg or 2.5 mg/kg). It can be seen from FIG. 4 that a single dose of drug resulted in significant reductions in weight gain. On average, rats gained weight during vehicle conditions (7.3±1.4 g over 10 days) and lost weight or failed to gain weight during the 1.25 mg/kg AM-251 (2.5±2.0 g) and 2.5 mg/kg (−5.1±2.1 g) conditions. Interestingly, in animals given the lowest dose of AM-251 (1.25 mg/kg), the duration of weight loss exceeded the duration of reduced food intake.

### EXAMPLE 3

### Effect of N-(Piperidin-1-yl)-5-(4-iodophenyl)-1-(2,4-dichlorophenyl)-4-methyl-1H-pyrazole-3-carboxamide (AM-251) when Administered Every 5 Days

To examine the effect of AM-251 given every 5 days, on food intake and body weight, 12 rats weighing between 440-540 g were fed Vanilla flavoured Ensure Plus® liquid diet (Abbott Laboratories, Abbott Park, Ill., U.S.A). Ensure is a highly palatable, nutrient dense, complete meal replacement composed of 53.3% carbohydrate, 29% fat, and 16.7% protein (1.41 kcal/g). Food was available for 17 h each day starting at 16:00 h. Food and water were presented in inverted glass bottles that were attached to the outside of the cage in order to minimize spillage. Rats were injected daily at 15:45 h with vehicle or every five days with AM-251 (5 mg/kg) (465.3±9.1 g, n=6). Food intake and body weight measurements were recorded daily.

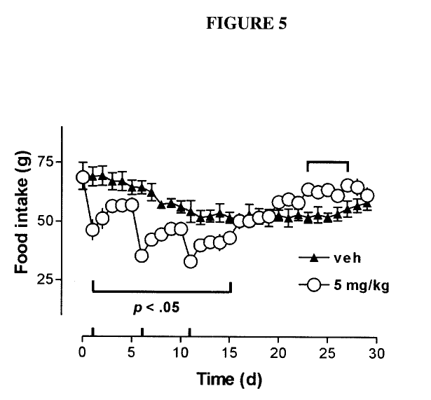

As can be seen in FIG. 5, AM-251 significantly reduced food intake in 5 day treated rats compared with vehicle treated controls, F_{(2,15)}=24.33, p<0.0001. There was also a significant treatment by time interaction, F_{(58,15)}=5.93, p<0.0001. The source of the interaction was investigated by comparing differences between vehicle and AM-251 treated rats at each time point using a one-way analysis of variance, and Newman-Keuls multiple comparison tests.

Reductions in food intake were significant for 4 days after the first dose of AM-251 (5 mg/kg) in the 5 day dosing schedule, compared with vehicle treated controls, p<0.05. Food intake was significantly reduced in rats treated every 5 days with AM-251 on days 1-4, and days 6-15, and the results thus show that reductions in food intake continued for five days after the last treatment was given in the 5 mg dosing strategy. Consistent with previous findings (Hildebrandt et al., 2003; Vickers et al., 2003, “Preferential Effects of the Cannabinoid CB1 Receptor Antagonist, SR 141716, on Food Intake and Body Weight Gain of Obese (fa/fa) Compared to Lean Zucker Rats”, Psychopharmacology (Berl) 167(1):103-11.), rats treated with AM-251 became significantly hyperphagic shortly after treatment ended, F_{(58,15)}=5.93, p<0.0001. Overeating relative to vehicle treated rats was significant on day 21 as well as during days 23-28 in rats with 5-day dosing schedules, p<0.05.

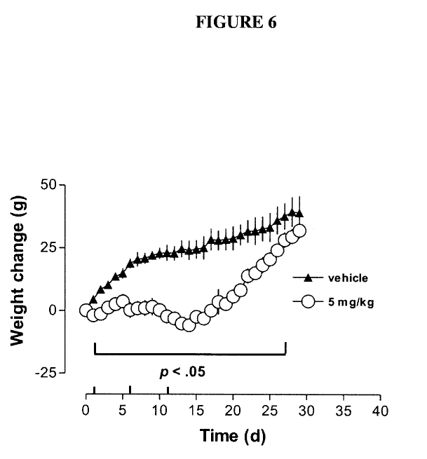

FIG. 6 shows that differences in weight change between vehicle and AM-251 treated rats were significant from day 1 to day 24, p<0.05, Newman-Keuls multiple comparison test. A 2-way ANOVA performed on the cumulative weight change data showed that differences in weight change between vehicle and AM-251 treated rats were significant between groups (F_{(29,15)}=28.4, p<0.0001) and over time, (F_{(29,15)}=38.2, p<0.0001). The difference in weight gain in vehicle treated rats during the first 15 days of the study also created a significant treatment by time interaction, F_{(58,15)}=5.85, p=0.0049. Note that reductions in weight gain were significant in AM-251 treated rats from day 1 until day 27, p<0.05, unpaired t-test, even though the last administration of AM-251 was given on day 11.

### EXAMPLE 4

### Comparison of Body Weight with Administration of Rimonabant or AM-251 Twice-Weekly on Two Consecutive Days

The present study was undertaken to examine the effects of infrequent doses of two CB₁ cannabinoid antagonists, rimonabant and AM-251, on the change in body weight when compared with daily delivery, which is common practice for the delivery of most pharmaceutical products. By delivering 1.5 times the normal dose of drug for 2 consecutive days instead of the usual daily dose on each of 7 days, the total weekly dose for a 2-day delivery would be less than half the dose for the rats receiving daily doses. This reduction in dose would not only expose the body to less of the drug but would also provide the drug infrequently so that long-term adaptation to the drug would be less likely to occur.

Another objective was to determine whether the animals were responding negatively to the drug as determined by the use of a two-flavored conditioned aversion test in which the drug was repeatedly paired with one flavor of liquid diet while the vehicle injection was paired with the other flavor. There were ten training trials before the test procedure was done so the rats had sufficient time to make associations between flavors and internal consequences. This test procedure involved giving the rats two bottles, containing the liquid diets flavored with chocolate and strawberry, and determining the individual rat's preference for one or the other of the flavors. A conditioned aversion was established if the rat chose against the flavor associated with the drug injections.

The doses of drugs used in these experiments were chosen to be the approximate rat equivalent of the doses used in human clinical trials. These dose levels would minimize side effects and would be less likely to produce discomfort, as would be indicated by the presence of a conditioned aversion to a flavored food paired with the drug delivery.

All rats in these experiments had surgery for the insertion of an intragastric catheter. This catheter allowed delivery of the drug directly into the stomach just before feeding without causing the trauma of intragastric gavage that is common in the experiments in the literature. The catheter (silicone rubber tubing 0.04 in id×0.85 in od, Medical Grade Silastic tubing, Dow Corning) was run from the back of the neck under the skin into a stab wound along the greater curvature of the stomach. A purse string suture held the tube tight within the stomach and a small piece of hernia mesh allowed the stomach, the tube and abdominal wall to seal strongly together and to prevent any leakage of gastric contents into the abdomen or under the skin. The inner bore of the tube was sealed with a hooked stainless-steel wire plug that could be removed before injection of the drug or vehicle was to be performed. Injection of the drug was followed by the insertion of a small amount of saline to deliver the full dose of drug and to keep the tube filled and open between injections.

All surgeries were done under ketamine-xylazine anesthetic and were followed by delivery of the antibiotic, Baytril (enrofloxecine), and repeated delivery of the analgesic, buprenorphine, to reduce the pain during recovery. The rats were allowed 10 days to recover from the surgery before beginning the adaptation phase of the experiment. While the rats were recovering from the surgery, they were put on a 17 hr feeding schedule of their powdered diet for 10-12 days. The rats were fed from 4 pm until 9 am the next morning. Water was available ad lib. The lights were turned off at 7 pm and on at 7 am the next morning.

The main period of feeding was during the night as is appropriate for nocturnal animals. Once the rats were fully recovered from surgery and adapted to this feeding schedule, the first baseline period of the experiment began. The rats weighed approximately 430 gm at the beginning of the experiment. All the rats were presented with the powdered laboratory chow every day at 4 pm and with the liquid diet, vanilla Ensure Plus (Ross Labs), on two “weekend” days to closely mimic the eating habits of many Western societies wherein the “weekend” diet may comprise more variety and/or an intake of higher caloric foods. During this time the rats were familiarized with the experimental procedure by injections of appropriate volumes of saline. This procedure continued for 14 days until intragastric drug delivery began. Once the rats had completed the two-week baseline adjustment period, the 32 male Lewis rats were assigned to one of five groups of rats based on their body weight and rate of weight gain. These assignments were made when the rats approached the time for the beginning of drug injections and were assigned in such a way as to have matched groups during the baseline period.

Each of the rats was assigned to one of five groups. There were 4 experimental groups of rats that included one of two drugs, AM-251 versus rimonabant, and one of two injection conditions, daily versus twice-weekly (n=5-7 in each group). These groups were compared to the group with daily injection of vehicle (n=8). The rats that had twice-weekly drug injections had vehicle injections on the 5 days when drug was not delivered so that all rats were injected with drug or vehicle on every day. After rats were assigned to each of the 5 groups, the flavor of the liquid diet of the rats was changed to either chocolate or strawberry in a balanced way so that half of the rats in each group would have chocolate flavor associated with the drug injection and the other half would have strawberry flavor associated with the drug injection. These flavors were presented to the rats for 17 hours directly after the drug injection so that the consequences of the injection would be associated in their minds with the primary flavor. In the middle of the week, the rats were given a 15 min exposure to the other secondary flavor just after a vehicle injection.

This conditioning procedure was done for 10 exposures before the actual conditioned aversion test was done on day 33. This aversion test was done by allowing each rat exposure for 17 hrs to two bottles, one containing chocolate-flavored and the other containing strawberry-flavored liquid diet. No drugs or vehicles were injected on this day. The rats were allowed to drink the diets for 15 min and the position of the nozzle was balanced across rats within each group to control for possible side preferences. Moreover, the rats were forced to taste both flavors for three or four licks before the diets were made freely available to them so the rats were fully aware that two flavors were present. Readings of the amount of food eaten were determined at 15 min (short-term) and at 17 hr (daily). The short-term data showed the rat's immediate response to taste with minimal internal signals from the ingestion of the diets that were the same in calories and composition. The 17-hour tests allowed repeated association between the internal consequences of the drug and the taste of the drug associated primary flavor.

The CB₁ cannabinoid antagonist drugs used in these experiments were AM-251 (Tocris) and rimonabant (Sanofi-Aventis product synthesized by the NIMH repository program). These drugs are fat-soluble and readily cross cell membranes. The appropriate amount of drug was dissolved DMSO (5% by weight) which was later mixed with Tween 80 (2% as an emulsifier) and made up to full weight (100%) with saline. The drug-solvent mixture was sonicated to obtain an even distribution of drug within the injection solution. The initial concentration of the daily dose of AM-251 was 0.80 mg/kg/day and of Rimonabant was 0.67 mg/kg/day. These daily doses were increased by 1.5× for the “weekend” dose. Both daily and weekend doses were increased again by 1.5× for the second 14 day period of drug injection. The amount of drug delivered was adjusted to the weight of each rat by changing the volume of the drug solution delivered.

An intragastric injection procedure was used in order to mimic the oral delivery used for most common drugs and necessary for the use of a pill to treat a long-term problem such as severe obesity. Once the rats had adapted to the feeding and lighting schedule, the experiment began.

The rats were weighed every day at 3:30 pm when they had been deprived of food for more than 6 hours and their weights would have been most stable. After the rats became familiar with the experimental procedures, their body weights were measured for a total of 42 days that were divided into 3 sets of 14 days each. During the first 14 days, the rats continued to adapt to the conditions of the experiment as well as having a daily intragastric injection of saline equal in volume/kg to their subsequent daily injection of drug. The drugs were injected 1-2 min before their daily food was presented to the rats. During the next 14 days, the rats were injected with the low dose of each of the cannabinoid drugs, and for the last 14 days, with the 50% higher dose.

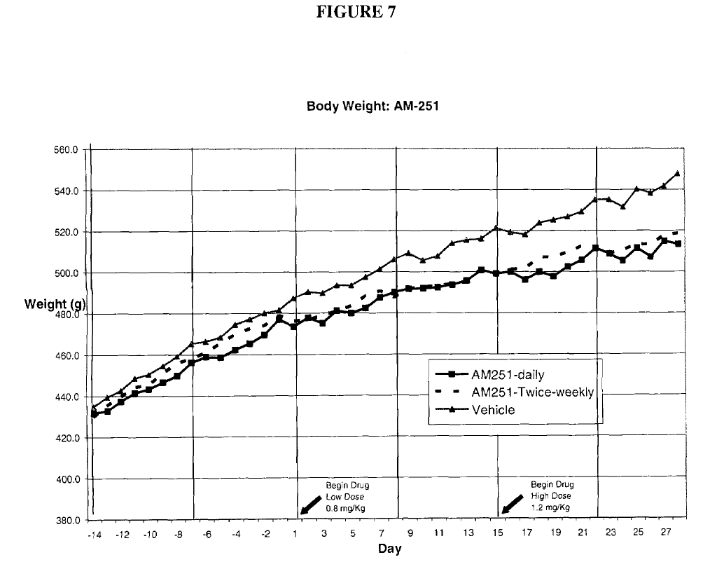

FIG. 7 illustrates the average daily body weight of male rats given daily or twice-weekly injections of the cannabinoid antagonist, AM-251. For the first 14 days (days −14 to −1), the rats were adapted to the conditions of the experiment which included daily 17-hour access to powdered laboratory chow, twice-weekly 17-hr access (“weekend days”) to the vanilla-flavored liquid diet, Ensure Plus, and ad lib access to water. During this acclimatization period, the rats had injections of saline just before eating to allow them to get used to the injection procedure. On day 1 of drug injection, the daily intragastric injection of 0.8 mg/kg of the drug, AM-251, began for one group of rats (n=7) and continued for 14 days. These rats also had a randomly assigned flavor of liquid diet, either chocolate or strawberry, fed to them for the first two days of injection. Another group of rats (n=6) were injected with a dose of 1.2 mg/kg for first two days and, then, with the vehicle for the next five days. This 2-day “weekend” injection procedure was repeated every week. The final group of rats (n=8) had an equal volume/kg of vehicle injected daily throughout the 28-day drug injection period. For the second two week injection period (days 15-28), the dose of AM-251 for both daily and weekend injections was increased by 50%, but otherwise the same procedures were followed.

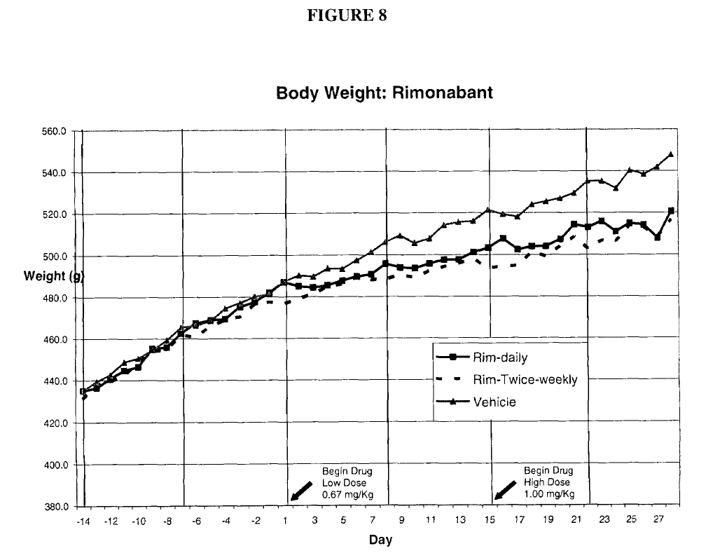

FIG. 8 illustrates the average daily body weight of male rats given daily or twice-weekly injections of the cannabinoid antagonist, rimonabant. The procedures were the same as those described in FIG. 7 except that the first daily dose of rimonabant was 0.67 mg/kg, which was equimolar with the above dose of AM-251. Drug doses were increased by 50% for twice-weekly injections and again by 50% during the third 14-day session.

With reference now to FIGS. 7 and 8, large and significant differences in body weight gain were observed when the rats were given the cannabinoid antagonists either daily or twice-weekly compared to the large weight gain of the controls with vehicle delivery. As expected from the matching procedure, the increases in body weight in the rats receiving the CB₁ receptor antagonists, AM-251 and rimonabant, and the vehicle were not significantly different during the pre-drug baseline period (F=0.21, p=0.93). The change in body weight for the 5 groups was also not significantly different during days 1-14 when the lower dose of each of the drugs was given (F=1.55, p=0.22), but was significant for days 15-28 (F=3.95, p<0.02) when the dose increased by 50%, and for the whole period of drug injection (days 1-28, F=3.82, p<0.02). When these 5 groups were combined across the two cannabinoid antagonists to make 3 groups, which doubled the size of the daily and twice-weekly groups, and compared them to the same vehicle group, the significance of the change in body weight was considerably increased (F=8.2, p<0.002). Specific comparisons between the four drug groups showed no significant differences (p>0.05) indicating that neither the type of drug nor the dosing procedure caused significant changes in body weight gain.

The conditioned aversion tests were divided into short-term (15 min) and daily food intake tests. The short-term percentage preference for the primary flavor associated with the delivery of AM-251 was 45% for the rats injected daily, 36% for the rats injected twice-weekly and 49% for the vehicle rats (F=0.18, p=0.84). For the rats injected with rimonabant, the corresponding percentages were 48%, 44% and 49%, respectively (F=0.002, p=0.98). These non-significant results were echoed in the daily intake tests. The percentage daily preference for the flavor associated with the delivery of AM-251 was 62% for the rats injected daily, 35% for the rats injected biweekly and 46% for the vehicle rats (F=1.9, p=0.18). For the rats injected with rimonabant, the corresponding percentages were 67%, 50% and 46%, respectively (F=0.87, p=0.44). None of the conditioned aversion tests were significant which suggests that the rats failed to make a negative association between the drug injection and the primary flavor that was repeatedly paired with the drug injection.

In summary, as shown in FIGS. 7 and 8, there were large, significant changes in body weight gain in the last 14 day period of drug injection and also in the total 28-day drug experiment. These results show that CB₁ receptor antagonists and inverse agonists are effective drugs in slowing body weight gain. Both rimonabant and AM-251 are effective in causing a significant slowing in weight gain in rats when delivered at low doses by the intragastric route. The most important observation is that these drugs cause about the same amount of weight loss over 28 days whether the drug is delivered daily at a low dose or less frequently (for only 2 out of 7 days) at a dose that was less than half this weekly dose. Thus, the overall weekly dose of both drugs and the frequency of drug delivery can be reduced and still retain a similarly reduced rate of weight gain. This biweekly drug delivery may reduce the adaptation that normally follows repeated administration of a drug.

The conditioned taste aversion paradigm showed that the rats did not develop an aversion to the flavor paired with the drug injection despite the fact that the rats had 10 pairings of the flavor with the drug over a full 17 hours before the one-day preference test was done on day 33. There were no significant differences in flavor preference noted at 15 minutes or at 17 hrs. The failure to obtain a conditioned taste aversion after 10 repeated full-day pairings of the drug with the primary flavor suggests that the side effects of these drugs at the doses given were relatively minor and insufficiently strong to lead to an avoidance of the flavors associated with the delivery of the drugs. In particular, the rats did not appear to distinguish between the two drugs or the dosing procedures on the conditioned aversion test.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. A method for the treatment of obesity, overweight and/or overeating in a mammal in need thereof comprising administering to said mammal a pharmaceutically effective amount of a CB₁ receptor antagonist as a unit daily dosage according to a continuous schedule having a dosing interval selected from the group consisting of once-weekly dosing, twice-weekly dosing, thrice-weekly dosing, biweekly dosing and bimonthly dosing.

58. A method according to claim 57 wherein said CB₁ receptor antagonist is a compound of the formula 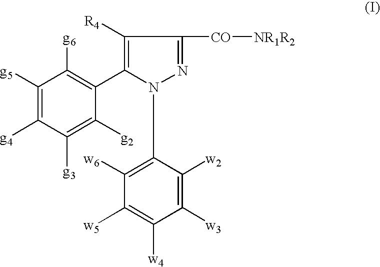
in which:
g₂, g₃, g₄, g₅ and g₆, and w₂, w₃, w₄, w₅ and w₆, are identical or different and are independently hydrogen, a chlorine, bromine or iodine atom, a (C₁-C₃)alkyl, a (C₁-C₃)alkoxy, a trifluoromethyl or a nitro group and g₄ can also be a phenyl group;
R₁ is a (C₁-C₆)alkyl or a hydrogen;
R₂ is —⁺NR₃R₅R₆ or —NR₅R₆;
R₃ is a (C₁-C₆)alkyl or R₃ forms a bridge with one of the atoms of the heterocyclic radical formed by NR₅R₆;
R₄ is hydrogen or a (C₁-C₅)alkyl; and
R₅ is hydrogen or a (C₁-C₆)alkyl and R₆ is hydrogen, a (C₁-C₆)alkyl, a phenyl or a (C₃-C₈)cycloalkyl, or R₅ and R₆, together with the nitrogen atom to which they are bonded, form a 5- to 10-membered saturated or unsaturated heterocyclic radical which is unsubstituted or monosubstituted or polysubstituted by a (C₁-C₆)alkyl, a benzyl, a phenyl, a hydroxyl, a (C₁-C₆)alkoxy or a halogen, with the proviso that if R₂ is NR₅R₆, R₅ and R₆, together with the nitrogen atom to which they are bonded, form a heterocyclic radical other than a 5- to 8-membered saturated radical which is unsubstituted or substituted by a (C₁-C₃)alkyl, a hydroxyl or a benzyl,
its salts or solvates.

59. A method according to claim 58 wherein the CB₁ receptor antagonist is N-(piperidin-1-yl)-5-(4-chlorophenyl)-1-(2,4-dichlorophenyl)-4-methyl-1H-pyrazole-3-carboxamide, one of its pharmaceutically acceptable salts or one of its solvates.

60. A method according to claim 58 wherein the CB₁ receptor antagonist is N-(piperidin-1-yl)-5-(4-iodophenyl)-1-(2,4-dichlorophenyl)-4-methyl-1H-pyrazole-3-carboxamide, one of its pharmaceutically acceptable salts or one of its solvates.

61. A method according to claim 59 wherein the dosing interval is once-weekly.

62. A method according to claim 59 wherein the dosing interval is twice-weekly or thrice-weekly.

63. (canceled)

64. A method according to claim 62 wherein the unit daily dosage is given on consecutive days.

65. A method according to claim 57 wherein the total weekly or monthly amount of the CB1 receptor antagonist given is reduced by at least 50% relative to a daily dosing interval without significantly changing the amount of body weight lost.

66. A method according to claim 59 wherein the unit daily dosage is given prior to or during a period of increased caloric intake.

67. (canceled)

68. A method for reducing food intake and/or body weight in a mammal comprising administering to said mammal a pharmaceutically effective amount of a CB₁ receptor antagonist once every 2 to 14 days.

69. A method according to claim 68 wherein said CB₁ receptor antagonist is a compound of the formula 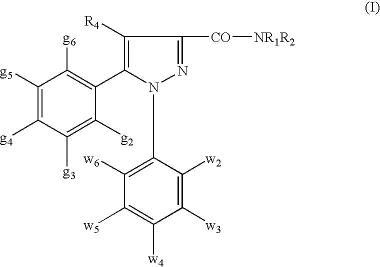
in which:
g₂, g₃, g₄, g₅ and g₆, and w₂, w₃, w₄, w₅ and w₆, are identical or different and are independently hydrogen, a chlorine, bromine or iodine atom, a (C₁-C₃)alkyl, a (C₁-C₃)alkoxy, a trifluoromethyl or a nitro group and g₄ can also be a phenyl group;
R₁ is a (C₁-C₆)alkyl or a hydrogen;
R₂ is —⁺NR₃R₅R₆ or —NR₅R₆;
R₃ is a (C₁-C₆)alkyl or R₃ forms a bridge with one of the atoms of the heterocyclic radical formed by NR₅R₆;
R₄ is hydrogen or a (C₁-C₅)alkyl; and
R₅ is hydrogen or a (C₁-C₆)alkyl and R₆ is hydrogen, a (C₁-C₆)alkyl, a phenyl or a (C₃-C₈)cycloalkyl, or R₅ and R₆, together with the nitrogen atom to which they are bonded, form a 5- to 10-membered saturated or unsaturated heterocyclic radical which is unsubstituted or monosubstituted or polysubstituted by a (C₁-C₆)alkyl, a benzyl, a phenyl, a hydroxyl, a (C₁-C₆)alkoxy or a halogen, with the proviso that if R₂ is NR₅R₆, R₅ and R₆, together with the nitrogen atom to which they are bonded, form a heterocyclic radical other than a 5- to 8-membered saturated radical which is unsubstituted or substituted by a (C₁-C₃)alkyl, a hydroxyl or a benzyl,
its salts or solvates.

70. A method according to claim 69 wherein the CB₁ receptor antagonist is N-(piperidin-1-yl)-5-(4-chlorophenyl)-1-(2,4-dichlorophenyl)-4-methyl-1H-pyrazole-3-carboxamide, one of its pharmaceutically acceptable salts or one of its solvates.

71. A method according to claim 69 wherein the CB₁ receptor antagonist is N-(piperidin-1-yl)-5-(4-iodophenyl)-1-(2,4-dichlorophenyl)-4-methyl-1H-pyrazole-3-carboxamide, one of its pharmaceutically acceptable salts or one of its solvates.

72. A method according to claim 68 wherein said mammal is a human or a companion animal such as a dog or a cat.

73. (canceled)

74. A method according to claim 68 wherein said CB₁ receptor antagonist is administered once every 2, 3 or 4 days.

75. A method according to claim 68 wherein said CB₁ receptor antagonist is administered once, twice or thrice every 5, 6 or 7 days.

76. A method according to claim 68 wherein said CB₁ receptor antagonist is administered once, twice or thrice every 7 days prior to or during an anticipated period of increased caloric intake.

77. A method for preventing overeating and/or inducing weight loss in a mammal in need of such treatment comprising repeatedly administering to said mammal a pharmaceutically effective amount of a CB₁ receptor antagonist for two or three consecutive days followed by a rest period of two to twelve days.

78. A method of claim 77 wherein the CB₁ receptor antagonist is N-(piperidin-1-yl)-5-(4-chlorophenyl)-1-(2,4-dichlorophenyl)-4-methyl-1H-pyrazole-3-carboxamide or N-(piperidin-1-yl)-5-(4-iodophenyl)-1-(2,4-dichlorophenyl)-4-methyl-1H-pyrazole-3-carboxamide, one of its pharmaceutically acceptable salts or one of its solvates.

79. A method of claim 78 wherein the two or three consecutive days precedes or is administered during a period of increased caloric intake.
